(19) United States
(12) Patent Application Publication
(10) Pub. No.: US 2014/0275952 A1
(43) Pub. Date: Sep. 18, 2014

(54) TREATMENT PLANNING FOR LUNG VOLUME REDUCTION PROCEDURES

(71) Applicant: Vida Diagnostics, Inc., Coralville, IA (US)

(72) Inventors: William S. Monroe, Iowa City, IA (US); Philippe Raffy, Edina, MN (US); Susan M. Wood, Palo Alto, CA (US)

(73) Assignee: Vida Diagnostics, Inc., Coralville, IA (US)

(21) Appl. No.: 14/209,451

(22) Filed: Mar. 13, 2014

Related U.S. Application Data
(60) Provisional application No. 61/782,308, filed on Mar. 14, 2013.

(51) Int. Cl. A61B 19/00; A61B 17/00

(52) U.S. Cl. CPC A61B 19/50; A61B 17/00234; A61B 19/5225; A61B 19/5212
USPC 600/407; 606/130

(57) ABSTRACT

Methods and systems for planning a bronchoscopic lung volume reduction procedure, such as placement of a one-way valve or biosealant or energy delivery in a patient's lungs. The system may include a processor and programming operable on the processor for planning the lung volume reduction procedure. Planning the lung volume reduction procedure by the processor may include receiving patient volumetric images, analyzing the images to identify the lobes and airway tree of the lungs, displaying a three dimensional model of the lungs, generating and displaying a suggested treatment volume on the three dimensional model, receiving a selected treatment volume from a user, generating and displaying a suggested treatment location within the airway tree, receiving a selected treatment location within the airway tree from the user, receiving a selected treatment modality from the user, and displaying a treatment plan.

### CROSS REFERENCE TO RELATED APPLICATION

This application claims priority to U.S. patent application Ser. No. 61/782,308, filed Mar. 14, 2013 and titled “TREATMENT PLANNING FOR LUNG VOLUME REDUCTION PROCEDURES.” The entire content of this application is incorporated herein by reference.

### BACKGROUND OF THE INVENTION

Severe emphysema is a debilitating disease that limits quality of life of patients and represents an end state of Chronic Obstructive Pulmonary Disease (COPD). It is believed that 3.5 million people in the US have the severe emphysematous form of COPD, and it is increasing in both prevalence and mortality. Current treatment methods for severe emphysema include lung volume reduction (LVR) surgery, which is highly invasive, and can be risky and uncomfortable for the patient. New treatment methods for treating emphysema include bronchoscopy guided lung volume reduction devices that aim to close off ventilation to the diseased regions of the lung, but maintain ventilation to healthier lung. Bronchoscopy-guided techniques have the promise to be less invasive, less costly and more highly accurate treatments for patients with severe disease and improve the quality of life of severe emphysema patients.

Emphysema can present itself in various disease forms (i.e., phenotypes). Predicting the right treatment for these patients at the appropriate time in the disease process may depend on the phenotype of the disease. Imaging techniques provide an in-vivo mechanism to objectively quantify and characterize disease phenotype and can be used as the patient selection process for the various procedural options. Quantitative imaging biomarkers can be used to effectively phenotype disease and therefore predict those patients most likely to respond to the targeted treatment options. By triaging the right patient to the appropriate therapy, there exists a greater promise for a positive impact on patient outcome, reduced healthcare costs, and replacing more invasive procedures like LVR surgery in treating patients with severe emphysema.

Bronchoscopic procedures such as the placement of pulmonary valves and the use of bio-sealants and energy delivery for lung volume reduction can provide effective ways of treating emphysema by shrinking overinflated portions of the lungs. However, because of the complexity of lung anatomy and the diversity of disease among individuals, planning for such procedures can be difficult. For example, it can be difficult to determine which locations are best suited for the placement of valves and whether how such locations can be best accessed bronchoscopically. Difficulties can therefore arise after such a treatment is already in progress, such as difficulties in accessing the location for placement of the valve or delivery of the bio-sealant or energy, or the results of such treatment may be less effective than anticipated due to disease aspects that might not have been appreciated before the procedures such as fissure integrity and the presence of collateral ventilation.

### SUMMARY

Certain embodiments of the present invention are described in the following illustrative embodiments. Various embodiments include systems and methods for planning a lung volume reduction procedure or for monitoring various aspects of a patient's lungs, such as after a lung volume reduction procedure. The treatment planning and monitoring may include three dimensional images of the patient's lungs created using patient volumetric images and which the clinician may use for a visual analysis of the plan or condition. The clinician may interact with the system to input data or select the type of information or model to be displayed as well as to select various aspects of the treatment plan.

In some embodiments, there is a system for planning a bronchoscopic lung volume reduction procedure for a patient. The system may include a processor and programming operable on the processor for planning the lung volume reduction procedure. Treatment planning may include receiving patient data comprising volumetric images of the patient, analyzing the volumetric images to identify lobes and airway tree of the lungs, displaying a three dimensional model of the patient's lungs, generating and displaying a suggested treatment volume on the three dimensional model, receiving a selected treatment volume from a user, generating and displaying a suggested treatment location within the airway tree, receiving a selected treatment location within the airway tree from the user, receiving a selected treatment modality from a user, and displaying a treatment plan. The treatment modality may include a one-way valve, an energy delivery therapy, or a bio-sealant, for example. The suggested treatment volume may be a lobe of the lungs, or a sub-lobe of the lungs, for example.

In some embodiments, planning the lung volume reduction procedure may further include generating a suggested treatment device. Planning the lung volume reduction procedure may also further include displaying information relating to the proposed treatment location. For example, the information may include one or more of an airway wall thickness, an airway diameter, and a length of airway having no branches. In some embodiments, planning the lung volume reduction procedure further includes receiving instructions from a user to move the suggested treatment location to a new location and displaying the new treatment location on the three dimensional model. In some embodiments, planning the lung volume reduction procedure further includes analyzing the patient data by comparing the patient data to a set of metrics to determine whether the patient is excluded from one or more modalities of lung volume reduction. The metrics may include one or more of a measure of emphysema score, heterogeneity, bronchiectasis, and fissure integrity, for example.

In some embodiments, the treatment plan includes a display of the three dimensional model of the patient's lungs in which a portion of the lung affected that would be affected by the selected treatment modality is highlighted and wherein the selected treatment location is highlighted. In some embodiments the treatment plan further includes a virtual bronchoscopy.

In some embodiments, the system is for monitoring the results of a lung volume reduction procedure for a patient and includes a processor and programming operable on the processor for displaying the results of a volume reduction procedure. Displaying the results of the lung volume reduction procedure may include accessing patient data including a first set of volumetric images of the patient from a first time and a second set of volumetric images from a second time later time, analyzing the first and second sets of volumetric images to identify lobes and airway tree of the lungs, and displaying a three dimensional model of the patient's lungs at the first time and at the second time. The time may be prior to a lung volume reduction procedure and the second time may be after the lung volume reduction procedure. In the three dimensional model of the patient's lungs includes, each lobes of a lung may be presented in a different color, for example. Displaying the results of the lung volume reduction procedure may also include calculating and displaying lung measurements at the first time and at the second time. The lung measurements may include one or more of a volume of one or more lobes; a heterogeneity score, a fissure integrity score, and/or a score for collateral ventilation. Displaying the results of a lung volume reduction procedure further include displaying a procedure date and/or a treatment modality or treatment device.

Still other embodiments include methods for treatment planning or patient monitoring. In some embodiments, the method is a method for planning a bronchoscopic lung volume reduction procedure using a treatment planning system comprising a processor, programming operable on the processor, and a user interface. The method may include inputting patient data including volumetric images of the patient, viewing a three dimensional model of the patient's lungs on the user interface, wherein the three dimensional model comprises a three dimensional model of an airway tree and lung parenchyma generated by the system and displayed on the user interface, viewing a suggested treatment volume generated by the system and displayed on the three dimensional model, selecting a treatment volume using the three dimensional model, viewing a suggested treatment location generated by the system and displayed on the airway tree, selecting a treatment location using the airway tree, selecting a treatment modality, and viewing a display of a treatment plan generated by the system and displayed on the user interface. In some embodiments, selecting a treatment modality includes selecting a one-way endobrochial valve, an energy delivery therapy, or a bio-sealant, for example. In some embodiments, the suggested treatment volume is a lobe of the lungs or a sub-lobe of the lungs.

The method may further include viewing a suggested treatment device generated by the system and displayed on the user interface. The method may further include viewing information relating to the proposed treatment location generated and displayed by the system on the user interface. The information may include one or more of an airway wall thickness, an airway diameter, and/or a length of airway having no branches, for example. The method may further include viewing an indication of whether the patient is excluded from one or more modalities of lung volume reduction, wherein the indication is generated by the system based on the patient data including the volumetric images and displayed on the user interface.

In some embodiments, the treatment plan includes the three dimensional model of the patient's lungs in which a portion of the lung affected that would be affected by the selected treatment modality is highlighted and wherein the selected treatment location is highlighted. The treatment plan may further include a virtual bronchoscopy.

### BRIEF DESCRIPTION OF THE DRAWINGS

The following drawings are illustrative of particular embodiments of the invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is a flowchart of a method of treatment planning in accordance with some embodiments;

FIG. 2 is screen shot of a user interface in accordance with some embodiments;

FIG. 3 is a device selection window user interface in accordance with some embodiments;

FIG. 4 is the device selection winder user interface after selection of a device by a user in accordance with some embodiments;

FIG. 5 is a screen shot of a user interface for treatment planning including a suggested treatment volume in accordance with some embodiments;

FIG. 5a is a screen shot of a user interface for treatment planning including a suggested treatment volume in accordance with some embodiments;

FIG. 6 is a screen shot of a user interface including user selection window for selecting type of process performed by system and type of treatment volume in accordance with some embodiments;

FIG. 7 is a screen shot of a user interface for treatment planning including suggested treatment locations in accordance with some embodiments;

FIG. 8a-8c are images of a user interface for treatment planning depicting adjustment of a treatment location by a user in accordance with some embodiments;

FIGS. 9a-9d are images of a user interface including a three dimensional model of a portion of the airway tree using different views in each figure to display different airway tree measurements in accordance with some embodiments;

FIG. 10 is a screen shot of a user interface for treatment planning including a treatment plan for placement of an intra-bronchial valve in accordance with some embodiments;

FIG. 11 is another screen shot of a user interface for treatment planning including a treatment plan for placement of an intra-bronchial valve in accordance with some embodiments;

FIG. 12 is another screen shot of a user interface for treatment planning including a treatment plan for placement of an intra-bronchial valve in which the airway diameter is shown in the display in accordance with some embodiments;

FIG. 13 is a screen shot of a user interface for monitoring the outcome of a lung volume reduction procedure;

FIG. 14 is a screen shot of a user interface for treatment planning including a treatment plan for delivery of a bio-sealant lung for volume reduction in accordance with some embodiments; and

FIG. 15 is a screen shot of a user interface for treatment planning including a treatment plan for delivery of energy for lung volume reduction in accordance with some embodiments.

### DETAILED DESCRIPTION

Embodiments described herein include systems for planning for interventional bronchoscopic treatment of pulmonary diseases such as emphysema. Because of the complexity of the lungs and the wide variety of ways in which lungs may be affected by pulmonary disease, as well as the anatomical differences amongst individuals, it can be difficult to plan interventional pulmonary treatments such as lung volume reduction treatments, such as valve placement, or the use of bioadhesives or energy modalities. The treatment planning system therefore provides a clinician with enhanced visualization and analysis of the lungs including assisting the physician with understanding the relationship between potential treatment devices and a patient's individual anatomy and disease characteristics. For example, the treatment planning system may utilize volumetric images or imaging data to analyze and identify patient anatomy and to present 3 dimensional models of the patient pulmonary anatomy to a clinician. A clinician may interact with the system to select a treatment location and treatment modality (e.g. type of valve) using the 3 dimensional model and/or 2 dimension patient images. The system may provide further analysis or guidance to the physician based on the images (e.g. patient anatomy) and clinician input (e.g. lung portion to be treated and device selection) in order to suggest locations for implementation of the treatment, such as the specific airway location for placement of a valve or for use of a bioadhesive or energy, for example, as well as how the clinician may access that location bronchoscopically. The suggestions provided by the system may be based on rules incorporated into the system, such as rules relating to physical restraints which may limit the options regarding treatment locations, such as the anatomical requirements for placement of a particular valve at a location or for navigation of a particular valve to a location.

The treatment planning system may include a processor, such as a processor in a computer, and may also include a visual display such as a monitor or other display screen. The system may also include instructions included in software, stored in memory of the system, and operable on the processor. The software may include instructions for the processor to perform the various steps and methods described herein, including instructions to receive patient data including volumetric imaging data, analyze the data, display images including three-dimensional images of the pulmonary tree, receive physician input, and analyze the pulmonary anatomy in light of the clinician input, and supply information to the clinician, and suggest treatment locations and approaches. In some embodiment, the treatment planning software may be incorporated into 3D pulmonary imaging software. In some embodiments, the treatment planning software and the 3D pulmonary imaging software may be separate software but may each be implemented by and/or incorporated into a common system. An example of 3D pulmonary imaging software that may be used in combination with the treatment planning software is the APOLLO quantitative pulmonary imaging system software available from VIDA Diagnostics, Inc.

The three-dimensional images or models of the lungs described herein are not truly created in three dimensions, because they exist on a flat two-dimensional visual display. Rather, the three-dimensional images described herein use perspective and shading, with the closest portions depicted in the foreground and more distant portions in the background, along with the ability of the user to rotate the images in some cases and/or to see multiple views, to show the entire 3-dimensional volume on the visual display. In contrast, each image in the series of the multi-dimensional volumetric images provided by a Multi-Planar Reconstruction (MPR) view, CT image, or MRI image, for example, is a two-dimensional planar image that depicts the tissue present in a single plane or slice. These images are typically presented in three orthogonal planes, which are referred to as the three orthogonal views and are typically identified as being axial, coronal and sagittal views.

Embodiments of the invention may allow the clinician to interact with the three-dimensional model of the lungs and the two-dimensional volumetric images associated with the 3-dimensional model. For example, the three-dimensional model and the associated two-dimensional images may be presented in a graphical user interface on a visual display. The user may interact with the graphical user interface, such as by selecting a button, icon, and/or one or more locations on the images or the model or elsewhere using a mouse, stylus, keypad, touchscreen or other type of interface known to those of skill in the art. The creation of the three-dimensional model may be performed by the system including a processor with software instructions to perform this function as well as software to permit a user to interact with the graphical user interface, to calculate and display desired data and images, and to perform the other functions described herein. The system may further include the visual display on which the graphical user interface is displayed. The three-dimensional model and two-dimensional images may be provided to a user (such as a clinician or researcher) as a graphical user interface on a visual display, which may be a computer screen, on which the images and data may be manipulated by the user.

Examples of the embodiments may be implemented using a combination of hardware, firmware, and/or software. For example, in many cases some or all of the functionality provided by examples may be implemented in executable software instructions capable of being carried out on a programmable computer processor. Likewise, some examples of the invention include a computer-readable storage device on which such executable software instructions are stored. In certain examples, the system processor itself may contain instructions to perform one or more tasks. System processing capabilities are not limited to any specific configuration and those skilled in the art will appreciate that the teachings provided herein may be implemented in a number of different manners.

As described above, the treatment planning system uses volumetric patient imaging to provide a platform for a clinician to plan interventional treatments for pulmonary disease. One example of the steps of a treatment planning procedure which may be performed by the treatment planning system is shown in the flowchart depicted in FIG. 1. However, it should be understood that the steps described herein need not necessarily all be performed or need not necessarily be performed in the order presented and various alternatives also exist.

The treatment planning procedure begins at the starting step 10 at which a clinician interacts with the system to direct it to begin a new treatment planning procedure. The clinician may select the volumetric patient volumetric images or imaging data to be used for the treatment planning procedure and the system may receive the volumetric images or imaging data in step 12 as well as other patient data. The volumetric patient images may be patient images or imaging data produced by CT scans, MRI scans, and/or PET scans, for example, from which a series of two-dimensional planar images (referred to herein as two-dimensional volumetric images or two-dimensional images) can be produced in multiple planes, for example. Other patient data which may be received by the system and which may be useful in the treatment planning process includes the patient's emphysema score, lung function test results such as FEV₁, and collateral ventilation measurements. For example, the amount of collateral ventilation may have been determined using a bronchoscopic system such as the CHARTIS System.

Next in step 14 the system analyzes the patient data. For example, the system may analyze the volumetric images to segment and identify the airways, the lobes, the sublobes, and the fissures, for example. Software for analyzing volumetric images of the lungs include 3D imaging software such as the Apollo quantitative pulmonary imaging software. Methods of identifying and characterizing sublobes are described in U.S. Pat. Pub. No. 2012-0249546, entitled Method and System for Visualization and Analysis of Sublobar Regions of the Lung, which is hereby incorporated by reference. Methods of identifying and characterizing the pulmonary fissures are described in U.S. Pat. App. No. 61/712,700, entitled Visualization and Characterization of Pulmonary Fissures, which is also hereby incorporated by reference. The methods used by the 3D pulmonary imaging software and the U.S. patent applications listed above may be likewise used to analyze the volumetric images for treatment planning as described herein.

After the system has analyzed the patient data, the system may create a graphical user interface in which a 3-dimensional model of the airways is presented along with other elements that may be used by the user during treatment planning. An example of such a graphical user interface is shown in the screenshot depicted in FIG. 2. The screenshot 100 includes a 3-dimensional model of the patient's lungs 102 constructed by the system from the volumetric imaging data. The upper lobes are displayed in a different color (represented by light gray) and demonstrate how the different lobes can be visualized. There is also a device selection window 104 and a device diagram window 106 which is empty because no device has been selected.

Next in step 16 the system may optionally perform a preliminary triage based upon the results of the analysis in step 14, for example. For example, the system may automatically apply a set of one or more metrics to determine if the patient is eligible or ineligible for LVR therapy. For example, bronchiectasis is a contraindication for many LVR procedures including valve placement procedures. The software may therefore automatically assess the patient data for the presence of bronchiectasis. If bronchiectasis or other contraindication is detected, its presence may be presented to the clinician and appropriate recommendations may be made by the system.

Various factors may be considered by the system for the patient triage step 16. These factors include the presence and degree of bronchiectasis, the integrity of the fissures, the heterogeneity of the emphysema (if present), and the emphysema score, if known. For each of these factors, whether or not LVR therapy is contraindicated may depend upon whether or not the factor is present or may depend upon the severity of the factor. For example, small amounts of bronchiectasis, fissure loss, or disease heterogeneity may be acceptable, but amounts beyond a certain cut off point may be contraindications to LVR therapy. The system may therefore compare the results of the analysis and the cut off points to determine whether or not a contraindication is present. Table 1 below provides an example of metrics which may be applied for patient triage in step 14 for various types of LVR therapy.

TABLE 1

| Treatment | Emphysema | Hetero- | Fissure | Bronchi- |
|---|---|---|---|---|
| modality | Score | geneity | Integrity | ectasis |
| Pulmonary valve | ≧40.0% | ≧15% | ≧78% | Not present |
| Energy | ≧40.0% | ≧15% | NA | NA |
| Bio-sealant | ≧40.0% | ≧15% | NA | NA |

In Table 1, it can be seen that certain factors may apply to all treatment modalities, such as emphysema score and disease heterogeneity. In each case, treatments are contraindicated unless the emphysema score is greater than or equal to 40%. However, other factors may apply to less than all of the treatment modalities, such as fissure integrity and bronchiectasis, which may only be considered for pulmonary valve placement but may not be considered for energy or bio-sealant treatment. Therefore, if pulmonary valve placement is contraindicated in a patient due to the presence of bronchiectasis or a fissure integrity score of less than 78%, for example, the patient may still qualify for energy or bio-sealant therapy if the other criteria are met. The triage results that are presented to a clinician may therefore include an indication of those modalities for which the patient might be eligible and/or those which are contraindicated. The triage results may also include numerical data or other information related to the metric (such as the patient's values and the cutoff values for some or all of the metrics) to indicate why a particular therapy modality was contraindicated or not and/or how close the patient was to the cut off values. It should be understood that the specific metrics and values shown in Table 1 are exemplary and other metrics and cut-off values may alternatively be used.

In step 18, device selection, the user may select the treatment modality to be used in the procedure being planned. The system may present the user with a plurality of modalities, such as a list of endobronchial valve placement, bio-sealant and/or energy delivery. The list of modalities may be limited to those modalities which were not contraindicated in step 16. A device schematic or other visual display may be presented or available to the user in association with specific devices. The user may select one of the presented treatment modality options in step 18.

An example of a graphical user interface for device selection by a user is shown in FIG. 3. In this figure, a user has clicked on the device selection dropdown icon 108 and a list 110 of treatment modalities appears beneath the treatment modality selection window 104. FIG. 4 shows the treatment modality selection window 104 after a user has made a device selection. The selected device, in this case an intrabronchial valve, is indicated in the treatment modality selection window 104. In addition, a diagram of the selected treatment modality appears in the device diagram window 106.

In some embodiments, the system may generate and display a suggested treatment volume in step 20. Although this step is shown in FIG. 1 as occurring after the treatment modality selection step 18, it may alternatively occur before the treatment modality selection step 18, for example. Based on the patient data analysis performed in step 14, the system may determine which lung volume or volumes for which treatment would be most likely to result in a positive response. This determination may be made by the system based on a set of factors or metrics which may vary depending upon the proposed treatment modality. An example of factors which may be considered by the system for determining whether or not to suggest a particular lung volume for a particular treatment is shown in Table 2 below.

TABLE 2

| Treatment | Emphysema | Hetero- | Fissure |
|---|---|---|---|
| modality | Score | geneity | Integrity |
| Pulmonary valve | ≧40.0% | ≧15% | ≧78% |
| Energy | ≧40.0% | ≧15% | NA |
| Bio-sealant | ≧40.0% | ≧15% | NA |

The factors presented in table 2 may be applied by the system to various lung volumes, such as lobes or sub-lobes, throughout the lungs to identify those volumes for which LVR therapy may be appropriate. The measurements of these factors may be used to derive simple rules or more sophisticated ones using fuzzy logic or decision tree techniques, for example. In some embodiments, the analysis may be automatic with a defined set of rules being applied. For example, the rules and cut-off values could be hard-coded (e.g. in an xml file). In other embodiments, the user may be able to select and/or modify the rules which are applied to the patient data. For example, the user may be able to adjust the cut off values which are applied by the system.

The recommended treatment volumes identified by the system may be presented to the user on the user interface using a 3 dimensional model of the lungs, for example. In some embodiments, the system may present the recommended volumes as recommended lobes for treatment or recommended sublobes. In some embodiments, the user may select which types of recommended volumes (lobes or sublobes) are displayed on the 3 dimensional model. The recommended lung treatment volumes may be distinguished from the remaining lung by presenting the recommended lung treatment volumes in a contrasting color or in some other manner to visually highlight the volume or volumes in comparison to the remainder of the lung. In addition, pertinent information may be provided to the user related to each recommended lung treatment volume. This information may appear on the user interface, such as close to or overlapping the recommended lung treatment volume on the 3 dimensional model, or may appear when a user interacts with the recommended lung treatment volume on the interface, such as when a user hovers a mouse over the volume. The information may include measurements such as the emphysema score, the heterogeneity score, and/or the fissure integrity score for the lung volume.

FIG. 5 is an example of a screenshot 100 of a graphical user interface including a 3-dimensional model of the lungs including two recommended treatment volumes 112. The recommended treatment volumes 112 may be shown in a contrasting color, for example. In FIG. 5, the recommended treatment volume is the right upper lobe as shown by the area of striping used to depict contrast with the remaining lung such as a contrasting color. Three 2-dimensional CT images 114, in each of the orthogonal views, are also included. Alternatively the CT images in this figure and in the other figures and embodiments described herein may be MPR views. A collateral ventilation input window 116 is also shown into which a user can enter a value for collateral ventilation for the recommended treatment location. A treatment volume information window 122 is shown in which the percent of the lung volume less than −950 H.U., the heterogeneity score (percent), the fissure integrity (percent) and the collateral ventilation are listed. FIG. 5a depicts an alternative screenshot with each of the sub-lobes visibly distinguished. In this example, the highlighted sub-lobe (represented by striping) is in contact with a missing portion of the fissure. There may be several sub-lobes touching a mission portion of the fissure on both sides of the fissure, as in the example shown in FIG. 5a. A visualization focusing on the sub-lobes can therefore give clinicians in indication of where the inter-lobar collateral ventilation may occur.

In FIG. 6, the graphical user interface also includes a user selection window 120 with which a user can select the type of procedure is being performed by the system, such as either a treatment planning or treatment monitoring procedure. The user may also select between the type of recommended treatment volume, such as either a lobe or a sublobe. A treatment volume information window 122 is also shown.

In step 22, the user selects the lung volume to be treated. The user may select a recommended treatment lung volume identified by the system in step 20 or may select a different lung volume. The user may select a treatment volume by clicking on the volume on the 3 dimensional model, for example. In some embodiments, the user may have the option to select between a treatment planning procedure and a patient monitoring and follow up procedure at the time of selecting the treatment volume. Alternatively, the selection between treatment planning or follow-up could be made at the start of the process, such as by using a similar window immediately before or after inputting the patient data.

After the treatment volume has been selected in step 22, the system may generate and display a proposed treatment plan in step 24. The treatment plan may include a proposed location or locations for implementation of the treatment modality, such as placement of one or more valves or for delivery of a bio-sealant or energy treatment. Based upon the previously selected treatment volume and the previously selected treatment modality, the system may identify a location or locations within the airway tree at which to implement the treatment (where to implant the valve, or apply the bio-sealant or energy treatment). The location may be selected by the system based on the patient anatomy as determined in the patient data analysis step, as well as the device dimensions and any other requirements related to the device, and the selected treatment volume. For example, in some embodiments, when a valve placement procedure is being planned, the system may identify one or more preferred location options that are the most proximal airways that are small enough to accept a valve and that will result in total occlusion of the selected treatment volume. Other factors which may be considered by the system include the length of the airways at the location not having an airway branch point. That is, a location may only be selected if the airways is sufficiently long relative to the size of the device and is without branch points along that length. A minimum wall thickness may also be required for an implantation site to be presented by the system to the user. A single location may be presented if treatment at that single location would result in occlusion of the entire selected treatment volume. Multiple locations may be presented, such as if a single treatment location is not adequate for occlusion of the entire treatment volume or if treatment at a single location would result in occlusion of lung outside of the selected treatment volume.

The proposed treatment location or locations may be displayed on the 3 dimensional model of the lungs, along with the selected lung treatment volume. This information may also be shown on 2-dimensional images, along with the 3 dimensional model, with identifying colors corresponding to the treatment locations and/or affected volume used consistently in each image. Alternatively, the selected lung volume and actual effected lung volume may be displayed on demand in association with a treatment location, such as if the user hovers over the proposed treatment location. In some embodiments, the proposed treatment locations may be listed, such as by the anatomical name of the proposed location.

The proposed pathway for bronchoscopically accessing the proposed treatment location or locations may also be provided with the proposed treatment plan in step 24. The proposed pathway may include a map showing bifurcations, airway diameters, and/or airway wall thickness, for example. Two-dimensional orthogonal views, such as CT scans, may be provided to the user, such that the user may zoom to the views to the target areas. Colors or other contrasting techniques may be used to indicate valve locations and corresponding valve sizes. For example, the proposed treatment location on the airway tree may be shown in a color, such as green, and the same color may be used to represent a valve size, such as a 7 mm diameter valve.

The proposed treatment plan may also include a specific recommended treatment device. In some embodiments, the system includes a set of rules for determining the appropriate size of a valve to use at a treatment location. For example, the system may use the factors listed in Table 3, below. Based on this analysis, the system may recommend a specific device and display that recommendation for the clinician. Information about the recommended device may also be displayed, such as the device dimensions and/or an image of the particular device. In some embodiments, the system may recommend a plurality of specific devices which would all qualify for use at the recommended treatment locations, for selection by the clinician.

TABLE 3

| | Wall | % | Centerline | Branch point |
|---|---|---|---|---|
| | Thickness | Occlusion | Length | angulation |
| Placement of | ≧1.0 mm | <90% | >.9.0 mm | NA |
| selected valve | | | | |
| Use next larger | <1.0 mm | NA | NA | NA |
| size valve | | | | |
| Use no valve | NA | ≧90% | NA | NA |
| Possibly anchor | NA | NA | <9.0 mm | NA |
| in branch point | | | | |
| Acceptable to | NA | NA | NA | ≧120 degrees |
| anchor in branch | | | | |
| point | | | | |

FIG. 7 is an example of a screenshot 100 of a display of a proposed treatment plan. The display includes a 3-dimensional model of the airway tree 124, with three proposed treatment locations 126 shown in a contrasting color represented by fine diagonal striping in the figure. Three orthogonal CT images 114 are also shown in the display. The screenshot also includes a list of the proposed treatment locations 128.

Next, the user selects the treatment plan in step 26. The user may select the treatment plan proposed by the system in step 24 in its entirety. Alternatively, the user may modify the proposed treatment plan, such as by selecting one or more alternative treatment locations, one or more alternative bronchoscopic pathways to the treatment locations and/or one or more treatment devices. In some embodiments, the user may be able to adjust the treatment location by moving the location proximally or distally in the same airway branch or to adjoining branches, such as by sliding the treatment location indicator proximally or distally on the 3 dimensional airway model or by otherwise adjusting the location using the graphical user interface. An example of a user adjusting a planned treatment location is shown in FIG. 8 in which a portion of the 3 dimensional airway model 126 is shown. In FIG. 8a, the initial treatment plan proposed by the system is presented to the user, with the proposed treatment locations 126 indicated in a contrasting color represented by fine diagonal stripes. In FIG. 8b, the user has selected a treatment location to be changed using a location change window 130. In FIG. 8c, the selected treatment location has been changed and has been moved more distally, beyond a branch, and replaced by two revised treatment locations 132, 134 shown in different contrasting colors represented by solid black and by black dots respectively. In some embodiments, the system may reanalyze the treatment location if it is adjusted by a user to determine the appropriate size of a valve to use at the new selected location, such as by using the metrics shown above in Table 3. The system may further display a new one or more recommended devices if appropriate given the change to the treatment location.

In some embodiments, once the treatment plan has been selected, the system may display and allow the user to review the selected treatment plan in detail in step 28. For example, in some embodiments, the system may provide a virtual bronchoscopy tool, which may include a listing of the corresponding measurements for each location. For example, one or more of the measurements shown in Table 3 may be provided for each treatment location. In addition, the angles for each airway junction may be displayed so that the user can assess accessibility of the treatment location. In some embodiments, the system may automatically play the bronchoscopy as an animation at the request of the user. The review may also allow the user to view the measurements for each treatment location in a simulation of the clinical environment. During the review, information that may be provided to the user includes multiple airway diameters, such as the airway diameter at the proximal most end of each treatment location, which will determine the size of the valve that may be used at the location, and the diameter at the distal most end of each treatment location. These two diameters can be used together to determine the relative sizes of the airway diameters to determine the presence and amount of tapering of the airway at the treatment location. This can be used to estimate the potential amount of settling that a valve may undergo. For example, if an airway narrows as it extends distally at a treatment location, a valve may be more likely to be seated properly than if it is straight or expands. Wall thickness may also be displayed to allow a user to estimate airway rigidity and elasticity, which may factor into proper valve placement. The airway length at the treatment location may also be provided, as it indicates the amount of space the clinician may have for placement of the valve. If this length is less than that of the valve to be placed, the valve may need to be placed into a bifurcation which may not be desired, or it may need to be changed to a different valve in order to obtain an appropriate fit. The distance to the treatment location, such as the distance from the main carina to the treatment location, may also be provided.

In some embodiments, the review may include options to change the view of the display. For example, the airways may be colored in a manner which is correlated to or highlights particular airway features such as the size (diameter) of the airways, the bifurcations, the wall thickness, occlusions, or airway length, to provide alternative ways to visually assess the treatment plan. An example of the alternate views is shown in the 3 dimensional airway model shown in FIGS. 9a-d, in which the bifurcations 134 are highlighted in FIG. 9a, the airway wall thickness is shown in a window 136 and correlated to different highlighted portions of the airway tree 124 in FIG. 9b; the points of occlusion 138 are highlighted in FIG. 9c, and the centerline length (the distance between branchpoints as if troweled by a bronchoscope) 140 is shown in window 140 and the corresponding portions of the airway tree are highlighted in FIG. 9d. Similarly portions of the airways are highlighted and correlated to window 142 to show the airway 144 diameters in the 3 dimensional model of the airway tree 124 shown in FIG. 12. It is useful to understand these aspects of the airways for treatment planning. The airway diameter is important for proper fit of the valve at the location and for navigating to the treatment location. Wall thickness can influence rigidity and elasticity and is therefore important for securing the valve at the treatment location.

An example of the step generating and displaying a selected treatment plan 28 for review by the user is shown in the screenshot 100 depicted in FIG. 10. In this embodiment, the user may the review tools by selecting them, such as by right clicking with a mouse, at the treatment location. The 3-dimensional model of the airway tree 124 includes the selected treatment locations 146 and a list 148 of information and measurement for each treatment location including the proximal diameter, airway length, anchor diameter, wall thickness and carina distance. Other measurements could also or alternatively be provided. A virtual bronchoscopy image 150 is also shown along with the bronchoscopy pathway 152 on the airway model 124. A ring 148 is shown in the 3D airway model encircling the airway and provides a marker which correlates to the position of ring 151 in the virtual bronchoscopy as well as an indication of the location for which airway measurements may be shown. A virtual axial cross section 154 of the bronchoscopy pathway through the airways is also shown.

Other treatment plan services screenshots 100 are shown in FIGS. 11 and 12. The displays shown in these figures are similar to the display in FIG. 10, but the airway tree 124 and associated information is presented differently. As noted previously, the airway tree 124 in FIG. 12 includes an indication of airway thickness in window 142. In FIG. 11, a window 141 is shown in which the clinician can select the types of views and airway information are displayed for the 3D airway model, such as selecting one of the views shown in FIGS. 9a-d. After the treatment plan is selected and reviewed, the selected treatment plan may be used to treat the patient by applying the selected treatment modality and selected treatment location for lung volume reduction by the clinician.

In some embodiments, the system may be used for monitoring a patient rather than for treatment planning. In some embodiments, the system can do both functions and the user selects the desired function, such as by selecting between treatment planning and monitoring at the start of the process. In a process in which patient monitoring is selected, the steps of receiving and analyzing patient data 12, 14 may be the same as for treatment planning. However, the patient data my include data, such as volumetric images, from both a first time point and a second time point, such as before and after a specific therapy such as placement of a valve. The patient data may then be used to generate information and visual displays comparing the status of the lungs at the two time points. In some embodiments, the system may already have patient data for the first time point stored in its memory. This patient data may have been input into the system and analyzed previously as part of a treatment planning procedure. The user may therefore direct the system to access this patient data from the first time point, and may only input additional patient data for the second time point into the system.

When used for patient monitoring or treatment follow up, the process begins similar to the process shown in FIG. 1. After starting the process, the system receives patient data from a first time and from a second time. The data includes volumetric images taken at the first time and the second time and may also include data such as fissure integrity scores, disease heterogeneity scores, lung function test results such as FEV, and collateral ventilation scores, for example, associated with the volumetric images at the first or second time. For example, the data may have been obtained at the same time or close in time to the volumetric images and the clinician may indicate that the data is associated with the set of images or the time point when inputting the data into the system. The system may then analyze the patient data as described above with regard to treatment planning. The system may then create a display for the user using the analyzed patient data. The display may include 3D models of the patient's lungs at the first and second time points including the airways, lung parenchyma and/or fissures and the lobes and/or sublobes may be distinguished or distinguishable, such as through the use of contrasting colors. Measurements relating to the lungs, as received by or calculated by the system, may be displayed for each time point. Volumes of the entire lung or portions therefore including lobes or sub-lobes may also be displayed for each time point as determined by the system using the volumetric images. When used for treatment follow up, such volume comparisons can provide an indication of treatment success.

Treatment monitoring includes evaluating a patient before and after a therapy. The therapy may be an endoscopic lung volume reduction procedure such as placement of a valve or delivery of a bio-sealant or energy to the lungs. Alternatively, it may be used to evaluate a patient before and after a lung volume reduction surgery. It may also be used to monitor disease progression in the absence of invasive treatments, such as disease progression without intervention, or to monitor the impact of non-invasive measures such as the use of or adjustment of pharmaceutical agents or smoking cessation.

An example of a screenshot of a patient monitoring display is shown in FIG. 13. The screenshot 100 includes 3 dimensional models of the lung parenchyma 160, with the lobes shown in distinct colors, both at a first time point (baseline, on the left) and a later second time (follow up, on the right) after a treatment. Likewise a 3 dimensional model of the airway tree is shown at the baseline time (left) and at follow up (right) treatment. In addition, information is provided on the display about the lungs at the baseline time and at follow up, such as the volume of each of the lobes. In this way, the changes in the lungs can be seen and understood by a user and monitored.

As mentioned above, various embodiments may be used for treatment planning for the use of bio sealants or energy based LVR procedures. The use of bio-sealants, such as the AERISEAL System from Aeris Therapeutics, is not as dependent upon airway morphology as valve placement. When the use of a bio-sealant is selected, measurements of the treatment locations (such as airway diameters) may not be necessary. Likewise, it may not be necessary to provide data regarding fissure integrity. Rather, lung volume measurements, such as the volume of a lobe or sublobe, along with the emphysema score and the heterogeneity of the lobe or sub-lobe, are important factors that may be determined by the system and used for treatment planning. The screenshot 100 for the treatment planning step of a bio-sealant procedure is shown in FIG. 14. The 3 dimensional model of the lungs 102 shows both the airway tree 124 and the parenchyma 160, with the treatment 148 volume 170 highlighted through the use of a contrasting color. The display also includes baseline measurements in window 162. Volumes of the lobes before and after treatment are shown in the baseline volume window 164 and the follow-up volume window 166. The baseline date 168 and follow-up date 170 are also shown. The total volume measurement displays the volume of the entire right or left lung. The tissue volume measurement displays the volume of the selected treatment volume. These measurements can be used by a clinician to help determine the appropriate amount of bio-sealant to use during the procedure. A CT image 114 of the lungs through the treatment volume is also shown, as well as a virtual bronchoscopy view 150 and a virtual axial cross section of the bronchoscopy pathway through airways leading to the treatment location.

Like bio-sealants, various forms of energy can be used to perform LVR therapy. Examples include the use of heated water vapor. Various embodiments can be used for treatment planning and dose determinations. Like bio-sealant treatments, energy based treatments are not dependent on airway morphology or fissure integrity and thus these measurements can be omitted from the display when energy treatment is being planned. However, important measurements such as volume measurements, emphysema score and heterogeneity may be determined by the system and provided on the display for the lobes, the sublobes, and/or for other treatment volumes. Again, the system may provide the treatment plan provided by the system may include the pathway to the treatment location, and this may be provided on the 3 dimensional airway model. Volume measurements which may be calculated by the system and displayed for the user include the total volume and the tissue volume, and these may be used to aid in determining the appropriate amount of energy to use during the procedure. The treatment plan may include and display the proposed order of treatment of the sublobes, such as by beginning with the smallest segments or lobes first and progressing to progressively larger lobes and sub-lobes. The system may also provide precise dosing recommendations as part of the treatment plan.

An example of a screenshot 100 for treatment plan review for energy delivery is shown in FIG. 15. It is similar to the other screenshots 100 for treatment plan review and includes a 3 dimensional model of the lungs 102 with the treatment volume highlighted, a corresponding CT image 114, a virtual bronchoscopy view 150 and a virtual axial cross section 154. The window 148 includes measurements for the treatment location including total volume, tissue volume, air volume, tissue to air ratio, and carina distance. Tissue volume and air volume are the volumes of the selected treatment volume comprised of tissue and of air, respectively, as may be determined by the system by analysis of the patient's volumetric images. These values, and their ratio, can be useful to a clinician for helping to determine the amount of energy needed to be delivered for the procedure. The device diagram window 106 displays an exemplary energy delivery device and energy is the selected modality in the device selection window 104.

When the treatment planning or follow up procedure is complete, the process ends at step 34.

In the foregoing detailed description, the invention has been described with reference to specific embodiments. However, it may be appreciated that various modifications and changes can be made without departing from the scope of the invention.

What is claimed is:

1. A system for planning a bronchoscopic lung volume reduction procedure for a patient comprising:
a processor;
programming operable on the processor for planning the lung volume reduction procedure;
wherein planning the lung volume reduction procedure comprises:
receiving patient data comprising volumetric images of the patient;
analyzing the volumetric images to identify lobes and airway tree of the lungs;
displaying a three dimensional model of the patient's lungs;
generating and displaying a suggested treatment volume on the three dimensional model;
receiving a selected treatment volume from a user;
generating and displaying a suggested treatment location within the airway tree;
receiving a selected treatment location within the airway tree from the user;
receiving a selected treatment modality from the user;
displaying a treatment plan.

2. The system of claim 1 wherein planning the lung volume reduction procedure further comprises generating a suggested treatment modality, the suggested treatment modality comprising a one-way valve, an energy delivery therapy, or a bio-sealant.

3. The system of claim 1 wherein the treatment plan comprises a display of the three dimensional model of the patient's lungs in which a portion of the lung affected that would be affected by the selected treatment modality is visually distinct and wherein the selected treatment location is visually distinct.

4. The system of claim 1 wherein planning the lung volume reduction procedure further comprises displaying information relating to the proposed treatment location.

5. The system of claim 4 wherein the information comprises one or more of an airway wall thickness, an airway diameter, and a length of airway having no branches.

6. The system of claim 1 planning the lung volume reduction procedure further comprising receiving instructions from a user to move the suggested treatment location to a new location and displaying the new treatment location on the three dimensional model.

7. The system of claim 1 wherein planning the lung volume reduction procedure further comprises analyzing the patient data comprising comparing the patient data to a set of metrics to determine whether the patient is excluded from one or more modalities of lung volume reduction, wherein the metrics include one or more of a measure of emphysema score, heterogeneity, bronchiectasis, and fissure integrity.

8. The system of claim 1 wherein the suggested treatment volume is a lobe of the lungs

9. The system of claim 1 wherein the suggested treatment volume is a sub-lobe of the lungs.

10. A system for monitoring the results of a lung volume reduction procedure for a patient comprising:
a processor;
programming operable on the processor for displaying the results of a volume reduction procedure;
wherein displaying the results of a lung volume reduction procedure comprises:
accessing patient data comprising first set of volumetric images of the patient from a first time and a second set of volumetric images from a second time later time, wherein the first time is prior to a lung volume reduction procedure and the second time is after the lung volume reduction procedure;
analyzing the first and second sets of volumetric images to identify lobes and airway tree of the lungs; and
displaying a three dimensional model of the patient's lungs at the first time and at the second time.

11. The system of claim 10 further comprising calculating and displaying lung measurements at the first time and at the second time, wherein the lung measurements include one or more of a volume of one or more lobes; a heterogeneity score, a fissure integrity score, and a score for collateral ventilation.

12. The system of claim 10 wherein displaying the results of a lung volume reduction procedure further comprises displaying a treatment modality.

13. A method for planning a bronchoscopic lung volume reduction procedure using a treatment planning system comprising a processor, programming operable on the processor, and a user interface, the method comprising:
inputting receiving patient data comprising volumetric images of the patient;
viewing a three dimensional model of the patient's lungs on the user interface, wherein the three dimensional model comprises a three dimensional model of an airway tree and lung parenchyma generated by the system and displayed on the user interface;
viewing a suggested treatment volume generated by the system and displayed on the three dimensional model;
selecting a treatment volume using the three dimensional model;
viewing a suggested treatment location generated by the system and displayed on the airway tree;
selecting a treatment location using the airway tree;
selecting a treatment modality; and
viewing a display of a treatment plan generated by the system and displayed on the user interface.

14. The method of claim 13 further comprising viewing a suggested treatment device generated by the system and displayed on the user interface prior to selecting a treatment modality.

15. The method of claim 13 wherein selecting a treatment modality comprises selecting a one-way endobrochial valve, an energy delivery therapy, or a bio-sealant.

16. The system of claim 13 wherein the treatment plan comprises the three dimensional model of the patient's lungs in which a portion of the lung affected that would be affected by the selected treatment modality is visually distinct and wherein the selected treatment location is visually distinct.

17. The system of claim 13 further comprising viewing information relating to the proposed treatment location generated and displayed by the system on the user interface, wherein the information comprises one or more of an airway wall thickness, an airway diameter, and a length of airway having no branches.

18. The system of claim 13 further comprising viewing an indication of whether the patient is excluded from one or more modalities of lung volume reduction generated by the system and displayed on the user interface.

19. The method of claim 13 wherein the suggested treatment volume is a lobe of the lungs

20. The method of claim 13 wherein the suggested treatment volume is a sub-lobe of the lungs.
